(19) United States
(12) Patent Application Publication
(10) Pub. No.: US 2008/0317752 A1
(43) Pub. Date: Dec. 25, 2008

(54) Inhibition of Tumorigenesis by Inhibition of a6b4 Integrin

(75) Inventor: Filippo G. Giancotti, New York, NY (US)

Correspondence Address: Marina Larson & Associates LLC;re: MSK, P. O. BOX 4928, DILLON, CO, 80435-4928, US

(73) Assignee: Sloan-Kettering Institute for Cancer Research, New York, NY (US)

(21) Appl. No.: 10/596,364

(22) PCT Filed: Apr. 18, 2006
(86) PCT No.: PCT/IB06/51199

§ 371 Date: Jun. 9, 2006

Related U.S. Application Data
(60) Provisional application No. 60/672,900, filed on Apr. 18, 2005.

(51) Int. Cl. A61K 39/395; A61K 31/7088; A61P 35/00

(52) U.S. Cl. 424/138.1; 514/44

(57) ABSTRACT

Inhibitors of a6b4 integrin that target beta 4 are used as therapeutic agents to inhibit tumorigensis in individuals, including humans, of tumors that express a6b4 integrin. The therapeutic agent may be an antibody or a small molecule, for example a laminin-5 analog, which binds to a6b4 integrin and inhibits its normal function. The therapeutic agent may also be a chemical species that interferes with the production of beta 4, including for example an antisense or RNAi species. The therapeutic agent is administered to the tissue or patient in a therapeutically effective amount. The therapeutic agent may be used as a single agent or in combination with other therapies, especially those directed toward suppressing the activity of RPTKs known to cooperate with a6b4, including but not limited to ErbB2, EGF-R, Met, and Ron.

### BACKGROUND OF INVENTION

This application relates to a method of inhibiting tumorigenesis, particularly in the case of tyrosine kinase-related cancers such as breast and prostate cancer, through the inhibition of a6b4 integrin using a therapeutic agant that targets the beta 4 portion of the integrin. In this application, the nomenclature a6b4 refers to the alpha-6-beta-4 integrin. Similar nomenclature with arabic or roman numerals is used for other integrins.

Integrins are a class of cellular transmembrane receptors known to bind to extracellular matrix proteins, and therefore they mediate cell-cell and cell-extracellular matrix interactions, referred generally to as cell adhesion events. The integrins connect the extracellular matrix to the intracellular cytoskeleton and cooperate with Receptor Protein Tyrosine Kinases (RPTKs) to regulate cell fate (Giancotti and Ruoslahti, 1999;

Hynes, 2003; Miranti and Brugge, 2002). Depending on the integrins they express and the matrix they attach to, normal cells proliferate or undergo growth arrest, migrate or remain stationary, and live or undergo apoptotic death. These effects imply that the integrins impart a stringent control to the action of RPTKs, determining the nature and direction of the cell's response to growth factors and cytokines (Giancotti and Tarone, 2003). Despite considerable amounts of cell biological data, genetic evidence of the significance of integrin signaling remains scarce. In particular, it has been difficult to separate the adhesive and signaling function of individual integrins in any model system analyzed to date.

The integrin receptors constitute a family of proteins with shared structural characteristics of noncovalent heterodimeric glycoprotein complexes formed of alpha and beta subunits. There are eight known beta subunits and fourteen known alpha subunits, which associate in various combinations to form twenty five receptors with different ligand specificities. The ligands for several of the integrins are adhesive extracellular matrix (ECM) proteins such as fibronectin, vitronectin, coflagens and laminin. It has been reported that the aVb1 integrin, a fibronectin receptor, and the aV integrins aVb3 and aVb5, which bind to several RGD-containing matrix proteins, promote aniogenesis. Hynes et al. (2002). This property has been considered as a basis for using inhibitors of such integrins as inhibitors of angiogenesis. See U.S. Pat. Nos. 5,981,478; 5,766,591; 6,358,970; and 6,645,991. However, while genetic experiments in mice have confirmed the role of a5b1 integrin in angiogenesis, they have not confirmed a role for the aV integrins, thus calling into question the efficacy of anti-angiogeneic therapy based on the latter group. Anti-angiogenic therapy based on inhibition of a5b1 integrin is problematic because of toxicity arising as a result of the critical involvement of this integrin adhesion of several cell types.

The a6b4 integrin is a laminin-5 receptor expressed by epithelial cells, Schwann cells, and endothelial cells and has several distinguishing features. The cytoplasmic domain of b4 is unusually long (ca. 1000 amino acids) and displays no homology to the short cytoplasmic tails of other b subunits. Upon a6b4 binding to matrix, the unique cytoplasmic domain of b4 is phosphorylated on multiple tyrosines by a Src Family kinase (SFK) and interacts directly with the signaling adaptor protein Shc, causing activation of the Ras to ERK cascade (Dans et al., 2001; Gagnoux-Palacios et al., 2003; Mainiero et al., 1995). In addition, the b4 tail mediates activation of PI-3K and Rac (Shaw, 2001; Shaw et al., 1997). Upon dephosphorylation, the cytoplasmic domain of b4 associates with the keratin cytoskeleton, causing assembly of hemidesmosomes and, hence, strengthening adhesion to laminin-5-containing basement membranes (Dans et al., 2001; Murgia et al., 1998; Spinardi et al., 1993). In contrast, the other integrins activate FAK/SFK signaling at focal adhesions (Geiger et al., 2001; Schlaepfer and Hunter, 1998) and, although some of them also recruit Shc, they do so by a distinct, indirect mechanism (Wary et al., 1998).

The pattern of expression of a6b4 in the skin is consistent with a role for a6b4 signaling in the control of epithelial proliferation. In normal epidermis, the expression of a6b4 is restricted to the basal cell layer, which comprises the rapidly dividing transit-amplifying cells (Borradori and Sonnenberg, 1999; Fuchs et al., 1997), while in skin diseases characterized by suprabasal proliferation, such as squamous carcinoma and psoriasis, a6b4 extends to the suprabasal layers (Pellegrini et al., 1992). In addition, ligation of a6b4 promotes progression through G1 and entry in S phase in keratinocytes treated with EGF, whereas ligation of a2b1 does not exert this effect (Mainiero et al., 1997).

Tumor biology studies have suggested a function for a6b4 signaling in tumor invasion. Many invasive carcinomas display elevated levels of a6b4 (Mercurio and Rabinovitz, 2001). Introduction of a6b4 in breast and colon carcinoma cells that have lost its expression activates PI-3K to Rac signaling and increases invasive ability in vitro (Shaw et al., 1997). In addition, it has been proposed that the b4 tail functions as an essential adapter and amplifier of pro-invasive signals elicited by activated Met in cells undergoing Met-induced oncogenesis (Trusolino et al., 2001). Finally, introduction of a dominant negative form of b4 impairs the survival of breast carcinoma cells, and this effect has been linked to the ability of mutant b4 to interfere with the assembly of hemidesmosomes and the establishment of a partially polarized phenotype (Weaver et al., 2002). Collectively, these results suggest the possibility that a6b4 promotes cell migration and invasion and confers resistance to apoptosis in carcinoma cells.

Commonly assigned U.S. Provisional Application No. 60/481,696, filed Nov. 22, 2003, and PCT application PCT/US2004/039189, which are incorporated herein by reference, described the use of a6b4 inegrin in controlling pathological neogenesis.

While this angiogenesis can occur in tumors, controlling angiogenesis is not the same as controlling or inhibiting tumorigenesis and tumor progression.

It has been shown previously that coexpression of a6b4 and laminin and amplification of ErbB-2 correlate with a poor prognosis in breast cancer patients (Slamon et al., 1987; Tagliabue et al., 1998). Although a significant fraction of human breast cancers show reduced expression of a6b4, a significant fraction of metastatic lymph nodes stain positive for a6b4 (Natali et al., 1992). Further, there is evidence that introduction of a6b4 increases the invasive ability of MDA-MB-435 breast carcinoma cells in in vitro assays (Shaw et al, 1997) and that the ability of b4-transfected MDA-MB-435 cells to metastasize to lung upon injection into a mouse tail vein requires the binding of a6b4 on cancer cells to Lu-ECAM-1 on lung endothelial cells. (Abdel-DGhany et al., 2001). Pathological studies have shown that the expression of a6b4 declines during prostate cancer progression (Cress et al., Cancer Metastasis Rev. 14:219-228, 1995). However, the integrin is still expressed at significant levels in Prostate Intraepithelial Neoplasia (PIN), and it may play a role at this stage of tumor progression. Nothwithstanding these findings, the role, if any, of a6b4 in tumor progression is not understood in the art. For example, whether the observed variations in expression levels are cause or effect, whether reduction changes I na6b4 have any actual impact of tumor growth or invasiveness in vivo, and how a6b4 interacts with other signaling moieties are not known.

### SUMMARY OF THE INVENTION

Following an investigation of the role of a6b4 in mice engineered to develop mammary tumors on expression of an activated version of ErbB-2 and on mice engineered to develop prostate cancer on expression of the SV-40 T Antigen, we have determined that a6b4 integrin signaling is necessary for the progression of breast and prostate cancer. This finding is also applicable to other tumor types that express a6b4, such as thyroid cancer, squamous carcinoma of the skin, cervix, and upper gastrointestinal tract, pancreatic cancer, colon cancer (Mercurio A M, Rabinovitz I, Towards a mechanistic understanding of tumor invasion—lessons from the alpha6beta 4 integrin. Semin Cancer Biol. 2001 April;11(2):12941). Thus, the present invention provides methods for the inhibition of tumorigenesis in tumors of this type using inhibitors of a6b4 integrin that target beta 4. In accordance with the method of the invention, an individual in whom tumorigensis is to be inhibited is exposed to a therapeutic agent effective to reduce the amount of active a6b4 integrin in the individual, at least at locations relevant to tumorigenesis. In one embodiment of the invention, the individual is a human patient. The therapeutic agent may be an antibody or a small molecule, for example a laminin-5 analog, which binds to a6b4 integrin and inhibits its normal function. The therapeutic agent may also be a chemical species that interferes with the production of beta 4, including for example an antisense or RNAi species. The therapeutic agent is administered to the tissue or patient in a therapeutically effective amount. The therapeutic agent may be used as a single agent or in combination with other therapies, especially those directed toward suppressing the activity of RPTKs known to cooperate with a6b4, including but not limited to ErbB2, EGF-R, Met, and Ron.

### BRIEF DESCRIPTION OF THE DRAWINGS

FIG. 1 shows a breeding strategy for introduction of MMTV-Neu^{NdI}-YD transgene into both wild-type and b4-1355T mice.

FIGS. 2A and B shows the extent of tumor free survival in b4 mutant and wild-type breed in accordance with the scheme in FIG. 1.

FIG. 3 shows the number of individual mammary tumors in individual mice.

FIG. 4 shows the growth of mammary tumors in wild-type and b4-mutant mice.

FIGS. 5A-C shows the difference in histological progression in mammary tumors in wild-type and b4-mutant mice.

FIG. 6 shows a breeding strategy for introduction of TRAMP into both wild-type and b4-1355T mice.

FIGS. 7A-D shows results of an MRI analysis indicative of tumor growth in b4-mutant and b4-wild-type TRAMP mice.

FIG. 8 shows survival of b4-mutant and b4-wild-type TRAMP mice.

FIG. 9 shows the sensitization upon loss of beta=4 signaling when MMTV-Neu (YD) mice bearing mammary tumors were treated with Iressa or vehicle (0.1% Tween-80).

FIGS. 10A and B shows reduction in tumor volume when MMTV-Neu (YD) mice bearing mammary tumors were treated with Iressa.

FIGS. 11A and B show differences in Ki-67+ cells in ducts/lobules (FIG. 11A) and MIN lesions (FIG. 11B).

FIG. 12 shows histological grading of tumor cells. Tumors isolated from 5-month old mice were subjected to H&E staining and examined microscopically. The graph shows the percentages of tumors in each catagory (WD: well differentiated; MD: moderately differentiated, PD: poorly differentiated) in Neu(YD)/b4-WT and Neu(YD)/b4-1355T mice.

FIG. 13 shows levels of spontaneous metastasis. Mice were sacrificed approximately 7.5 weeks after detection of their first palpable tumor. Sagittal lung sections (2-4 per mouse) were stained with H&E and examined microscopically. Mice were assigned to 3 groups depending on their average number of metastases per lung section (<1, 1-5, >5). The graph shows the percentage of mice of the indicated genotypes in each group. The P value was calculated by the Chi-squared test. The inset shows the mean cumulative tumor burden i SD in each cohort of mice at the time of euthanasia

FIGS. 14A and B show results for tumor growth in 3D Matrigel. Primary tumor cells (5×10³) were seeded in 3D Matrigel and cultured. Cellular structures were released from Matrigel by dispase and dissociated with trypsin. Cells were counted at the indicated times. The graph shows the average growth curves ±SEM (standard error of the mean) of Neu(YD)/b4-WT cells (W) and Neu(YD) b-1355T cells (T).

FIGS. 15A-C shows results when Neu-b4-WT and Neu-b4-1355T cells were cultured on collagen I in serum-free medium (FIG. 15A) or with 10% FBS (FIG. 15B) and counted at the indicated times.

The arrow indicates when cells reached confluence in FBS. In FIG. 15C, the indicated cells were cultured on collagen I under sparse conditions with serumfree medium (SFM) or 10% FBS (FBS) or at confluency with 10% FBS (FBS confluent) in the presence of BrdU for 24 or 28 hrs, respectively. The graph shows the percentage (mean ±SD) of BrdU+ cells.

FIG. 16 shows results when Neu-b4-WT and Neu-b4-1355T cells treated with Iressa (10 μM) or vehicle alone (DMSO) were subjected to Matrigel invasion assay in response to FBS. The graph shows the mean number of invaded cells ±SD per microscopic field in triplicate.

FIG. 17 shows results when Neu-b4-WT and Neu-b4-1355T cells were injected in the tail vein. Percentages of lung section areas occupied by metastases (mean ±standard deviation) were quantified 30 days later by image analysis with Metamorph software.

FIGS. 18A shows results when tumor-bearing Neu(YD)/b4-WT (b4-WT) and Neu(YD)/b4-1355T (b4-1355T) mice were treated with Iressa (100 mg/Kg/day) or vehicle control (0.1% Tween-80) for 24 days or with a single dose of Doxorubicin (10 mg/Kg). The graph shows the mean change in tumor volume ±SEM at day 24 for Iressa or vehicle alone and day 21 for Doxorubicin.

FIG. 18B shows results when tumor-bearing Neu(YD)/b4-WT (b4-WT) and Neu(YD)/b4-1355T (b4-1355T) mice were treated with Iressa (100 mg/Kg/day) or vehicle control (0.1% Tween-80) for at least 7 days. Tumor sections were stained by IHC with anti-Ki-67 followed by counterstaining with hematoxylin. Bar=100 μM. The graph shows the mean number of Ki-67+ cells±SD per field.

### DETAILED DESCRIPTION OF THE INVENTION

As used in this application, the term ‘tumorigenesis’ refers to initiation of primary or metastatic tumor growth, and the promotion of invasive growth.

As used in this application, the term ‘inhibition’ refers to a reduction of the event or activity inhibited to an extent sufficient to produce an observable result. Complete elimination of the event or activity is not required.

As used in this application, the term ‘amount of active a6b4 integrin’ refers to the observable tumorigenesis-promoting activity resulting from a6b4 integrin present in a tissue. Reductions in the amount of the active a6b4 integrin can result from a reduction in the amount of a6b4 integrin, i.e, effectively a reduction in concentration; a reduction in the capacity of individual molecules of a6b4 integrin to promote tumorigenesis, i.e. effectively a change in the quality of the integrin, or combinations thereof. The first type of reduction will most commonly be achieved by limiting the production of a6b4 integrin, for example using a antisense oligonucleotide or RNAi techniques, although it could also be achieved by accelerating the decomposition of a6b4 integrin. The second type of reduction is most readily achieved through physical binding of the integrin with a ligand that competes with the normal ligand for binding to the receptor.

As used in this application, the terms ‘treatment’ or ‘treating’ refer to the application of a therapeutic agent to achieve a reduction in the amount of active a6b4 integrin so as to produce a benefit to a patient being treated. Such a benefit need not be a complete or permanent cure, but may be only a lessening of the rate at which tumorigenesis is occurring, thereby delaying progression of a disease condition.

As used in this application, the term ‘administration’ refers to any means by which a therapeutic agent can be delivered to a tissue, including without limitation oral, nasal and transdermal administration and injection, for example subcutaneous, subdermal, intramuscular, intravenous, intrathecal or peritoneal injection. For treatment of eye-associated tumorgenesis, direct injection to the eye may be used. The therapeutic agent of the invention can be used in combination with other agents used in the treatment of cancer. In particular, the therapeutic agent of the invention is suitably used in combination with kinase inhibitors such as Iressa. Use in combination entails the administration of two or more agents in a time course where the effects of at least one of the agents is improved as a result of the use of the other. Two agents need no be administered at the same time to be considered use in combination, and may be used in any order.

The effective amount of a therapeutic agent to be administered varies depending on the nature of the therapeutic agent, and will frequently reflect a balancing of therapeutic benefits and side effects. However, the determination of specific amounts for a given therapeutic is routine and within the skill in the art.

Therapeutic agents useful in the present invention may be antibodies, aptamers or small molecules that bind to a6b4 integrin to produce a reduction in activity. Examples include small molecules which block b4 signaling by binding to b4, and have specific functions such as inhibiting nuclear translocation of NfkB. Where an antibody therapeutic agent is used, it may be administered in the form of the antibody, or formed in situ by expression of a nucleic acid sequence encoding an a6b4 integrin-specific antibody. Such antibodies may be monoclonal, polyclonal, or modified constructs, for example single chain Fv constructs, targeting a6b4 integrin. Binding sites may be on the alpha chain, the beta chain or both chains of the a6b4 integrin. Non-antibody binding proteins could also be employed. For example, human integrin-beta-4 binding protein is known and has the sequence:

(Seq. ID. No. 1)

MAVRASFENNCEIGCFAKLTNTYCLVAIGGSENFYSVFEGELSDTLPVVH
ASIAGCRIIGRMCVGNRHGLLVPNNTTDQELQHIRNSLPDTVQIRRVEER
LSALGNVTTCNDYVALVHPDLDRETEEILADVLKVEVFRQTVADQVLVGS
YCVFSNQGGLVHPKTSIEDQDELSSLLQVPLVAGTVNRGSEVIAAGMVVN
DWCAFCGLDTTSTELSVVESVFKLNEAQPSTIATSMRDSLIDSLT
(NM_002212).

The therapeutic agent may also be a nucleic acid that results in a reduction in the amount of active a6b4 integrin, for example an antisense oligonucleotide or an RNA molecule that works by an RNAi mechanism. The nucleic acid may target, via a sequence specific mechanism, the alpha chain or the beta chain. The coding sequence of the beta 4 chain of human integrin is known from NM_{—}000213 to be as follows:

| (Seq. ID. No.2) | |
|---|---|
| 1 | atggcagggc cacgccccag cccatgggcc aggctgctcc tggcagcctt gatcagcgtc |
| 61 | agcctctctg ggaccttggc aaaccgctgc aagaaggccc cagtgaagag ctgcacggag |
| 121 | tgtgtccgtg tggataagga ctgcgcctac tgcacagacg agatgttcag ggaccggcgc |
| 181 | tgcaacaccc aggcggagct gctggccgcg ggctgccagc gggagagcat cgtggtcatg |
| 241 | gagagcagct tccaaatcac agaggagacc cagattgaca ccaccctgcg gcgcagccag |
| 301 | atgtcccccc aaggcctgcg ggtccgtctg cggcccggtg aggagcggca ttttgagctg |
| 361 | gaggtgtttg agccactgga gagccccgtg gacctgtaca tcctcatgga cttctccaac |
| 421 | tccatgtccg atgatctgga caacctcaag aagatggggc agaacctggc tcgggtcctg |
| 481 | agccagctca ccagcgacta cactattgga tttggcaagt ttgtggacaa agtcagcgtc |
| 541 | ccgcagacgg acatgaggcc tgagaagctg aaggagccct ggcccaacag tgaccccccc |
| 601 | ttctccttca agaacgtcat cagcctgaca gaagatgtgg atgagttccg gaataaactg |
| 661 | cagggagagc ggatctcagg caacctggat gctcctgagg gcggcttcga tgccatcctg |
| 721 | cagacagctg tgtgcacgag ggacattggc tggcgcccgg acagcaccca cctgctggtc |
| 781 | ttctccaccg agtcagcctt ccactatgag gctgatggcg ccaacgtgct ggctggcatc |
| 841 | atgagccgca acgatgaacg gtgccacctg gacaccacgg gcacctacac ccagtacagg |
| 901 | acacaggact acccgtcggt gcccaccctg gtgcgcctgc tcgccaagca caacatcatc |
| 961 | cccatctttg ctgtcaccaa ctactcctat agctactacg agaagcttca cacctatttc |
| 1021 | cctgtctcct cactgggggt gctgcaggag gactcgtcca acatcgtgga gctgctggag |
| 1081 | gaggccttca atcggatccg ctccaacctg gacatccggg ccctagacag cccccgaggc |
| 1141 | cttcggacag aggtcacctc caagatgttc cagaagacga ggactgggtc ctttcacatc |
| 1201 | cggcgggggg aagtgggtat ataccaggtg cagctgcggg cccttgagca cgtggatggg |
| 1261 | acgcacgtgt gccagctgcc ggaggaccag aagggcaaca tccatctgaa accttccttc |
| 1321 | tccgacggcc tcaagatgga cgcgggcatc atctgtgatg tgtgcacctg cgagctgcaa |
| 1381 | aaagaggtgc ggtcagctcg ctgcagcttc aacggagact tcgtgtgcgg acagtgtgtg |
| 1441 | tgcagcgagg gctggagtgg ccagacctgc aactgctcca ccggctctct gagtgacatt |
| 1501 | cagccctgcc tgcgggaggg cgaggacaag ccgtgctccg gccgtgggga gtgccagtgc |
| 1561 | gggcactgtg tgtgctacgg cgaaggccgc tacgagggtc agttctgcga gtatgacaac |
| 1621 | ttccagtgtc cccgcacttc cgggttcctg tgcaatgacc gaggacgctg ctccatgggc |
| 1681 | cagtgtgtgt gtgagcctgg ttggacaggc ccaagctgtg actgtcccct cagcaatgcc |
| 1741 | acctgcatcg acagcaatgg gggcatctgt aatggacgtg gccactgtga gtgtggccgc |
| 1801 | tgccactgcc accagcagtc gctctacacg gacaccatct gcgagatcaa ctactcggcg |
| 1861 | atccacccgg gcctctgcga ggacctacgc tcctgcgtgc agtgccaggc gtggggcacc |
| 1921 | ggcgagaaga aggggcgcac gtgtgaggaa tgcaacttca aggtcaagat ggtggacgag |
| 1981 | cttaagagag ccgaggaggt ggtggtgcgc tgctccttcc gggacgagga tgacgactgc |
| 2041 | acctacagct acaccatgga aggtgacggc gcccctgggc ccaacagcac tgtcctggtg |
| 2101 | cacaagaaga aggactgccc tccgggctcc ttctggtggc tcatccccct gctcctcctc |
| 2161 | ctcctgccgc tcctggccct gctactgctg ctatgctgga agtactgtgc ctgctgcaag |
| 2221 | gcctgcctgg cacttctccc gtgctgcaac cgaggtcaca tggtgggctt taaggaagac |
| 2281 | cactacatgc tgcgggagaa cctgatggcc tctgaccact tggacacgcc catgctgcgc |
| 2341 | agcgggaacc tcaagggccg tgacgtggtc cgctggaagg tcaccaacaa catgcagcgg |
| 2401 | cctggctttg ccactcatgc cgccagcatc aaccccacag agctggtgcc ctacgggctg |
| 2461 | tccttgcgcc tggcccgcct ttgcaccgag aacctgctga agcctgacac tcgggagtgc |
| 2521 | gcccagctgc gccaggaggt ggaggagaac ctgaacgagg tctacaggca gatctccggt |
| 2581 | gtacacaagc tccagcagac caagttccgg cagcagccca atgccgggaa aaagcaagac |
| 2641 | cacaccattg tggacacagt gctgatggcg ccccgctcgg ccaagccggc cctgctgaag |
| 2701 | cttacagaga agcaggtgga acagagggcc ttccacgacc tcaaggtggc ccccggctac |
| 2761 | tacaccctca ctgcagacca ggacgcccgg ggcatggtgg agttccagga gggcgtggag |
| 2821 | ctggtggacg tacgggtgcc cctctttatc cggcctgagg atgacgacga gaagcagctg |
| 2881 | ctggtggagg ccatcgacgt gcccgcaggc actgccaccc tcggccgccg cctggtaaac |
| 2941 | atcaccatca tcaaggagca agccagagac gtggtgtcct ttgagcagcc tgagttctcg |
| 3001 | gtcagccgcg gggaccaggt ggcccgcatc cctgtcatcc ggcgtgtcct ggacggcggg |
| 3061 | aagtcccagg tctcctaccg cacacaggat ggcaccgcgc agggcaaccg ggactacatc |
| 3121 | cccgtggagg gtgagctgct gttccagcct ggggaggcct ggaaagagct gcaggtgaag |
| 3181 | ctcctggagc tgcaagaagt tgactccctc ctgcggggcc gccaggtccg ccgtttccac |
| 3241 | gtccagctca gcaaccctaa gtttggggcc cacctgggcc agccccactc caccaccatc |
| 3301 | atcatcaggg acccagatga actggaccgg agcttcacga gtcagatgtt gtcatcacag |
| 3361 | ccaccccctc acggcgacct gggcgccccg cagaacccca atgctaaggc cgctgggtcc |
| 3421 | aggaagatcc atttcaactg gctgccccct tctggcaagc caatggggta cagggtaaag |
| 3481 | tactggattc agggcgactc cgaatccgaa gcccacctgc tcgacagcaa ggtgccctca |
| 3541 | gtggagctca ccaacctgta cccgtattgc gactatgaga tgaaggtgtg cgcctacggg |
| 3601 | gctcagggcg agggacccta cagctccctg gtgtcctgcc gcacccacca ggaagtgccc |
| 3661 | agcgagccag ggcgtctggc cttcaatgtc gtctcctcca cggtgaccca gctgagctgg |
| 3721 | gctgagccgg ctgagaccaa cggtgagatc acagcctacg aggtctgcta tggcctggtc |
| 3781 | aacgatgaca accgacctat tgggcccatg aagaaagtgc tggttgacaa ccctaagaac |
| 3841 | cggatgctgc ttattgagaa ccttcgggag tcccagccct accgctacac ggtgaaggcg |
| 3901 | cgcaacgggg ccggctgggg gcctgagcgg gaggccatca tcaacctggc cacccagccc |
| 3961 | aagaggccca tgtccatccc catcatccct gacatcccta tcgtggacgc ccagagcggg |
| 4021 | gaggactacg acagcttcct tatgtacagc gatgacgttc tacgctctcc atcgggcagc |
| 4081 | cagaggccca gcgtctccga tgacactggc tgcggctgga agttcgagcc cctgctgggg |
| 4141 | gaggagctgg acctgcggcg cgtcacgtgg cggctgcccc cggagctcat cccgcgcctg |
| 4201 | tcggccagca gcgggcgctc ctccgacgcc gaggccccca cggccccccg gacgacggcg |
| 4261 | gcgcgggcgg gaagggcggc agccgtgccc cgcagtgcga cacccgggcc ccccggagag |
| 4321 | cacctggtga atggccggat ggactttgcc ttcccgggca gcaccaactc cctgcacagg |
| 4381 | atgaccacga ccagtgctgc tgcctatggc acccacctga gcccacacgt gccccaccgc |
| 4441 | gtgctaagca catcctccac cctcacacgg gactacaact cactgacccg ctcagaacac |
| 4501 | tcacactcga ccacactgcc cagggactac tccaccctca cctccgtctc ctcccacgac |
| 4561 | tctcgcctga ctgctggtgt gcccgacacg cccacccgcc tggtgttctc tgccctgggg |
| 4621 | cccacatctc tcagagtgag ctggcaggag ccgcggtgcg agcggccgct gcagggctac |
| 4681 | agtgtggagt accagctgct gaacggcggt gagctgcatc ggctcaacat ccccaaccct |
| 4741 | gcccagacct cggtggtggt ggaagacctc ctgcccaacc actcctacgt gttccgcgtg |
| 4801 | cgggcccaga gccaggaagg ctggggccga gagcgtgagg gtgtcatcac cattgaatcc |
| 4861 | caggtgcacc cgcagagccc actgtgtccc ctgccaggct ccgccttcac tttgagcact |
| 4921 | cccagtgccc caggcccgct ggtgttcact gccctgagcc cagactcgct gcagctgagc |
| 4981 | tgggagcggc cacggaggcc caatggggat atcgtcggct acctggtgac ctgtgagatg |
| 5041 | gcccaaggag gagggccagc caccgcattc cgggtggatg gagacagccc cgagagccgg |
| 5101 | ctgaccgtgc cgggcctcag cgagaacgtg ccctacaagt tcaaggtgca ggccaggacc |
| 5161 | actgagggct tcgggccaga gcgcgagggc atcatcacca tagagtccca ggatggagga |
| 5221 | cccttcccgc agctgggcag ccgtgccggg ctcttccagc acccgctgca aagcgagtac |
| 5281 | agcagcatca ccaccaccca caccagcgcc accgagccct tcctagtgga tgggctgacc |
| 5341 | ctgggggccc agcacctgga ggcaggcggc tccctcaccc ggcatgtgac ccaggagttt |
| 5401 | gtgagccgga cactgaccac cagcggaacc cttagcaccc acatggacca acagttcttc |
| 5461 | caaacttga |

The coding sequence of the alpha 6 chain of human integrin is known from

NM_{—}000210 to be as follows:

| (Seq. ID. No. 3) | |
|---|---|
| 1 | atggccgccg ccgggcagct gtgcttgctc tacctgtcgg cggggctcct gtcccggctc |
| 61 | ggcgcagcct tcaacttgga cactcgggag gacaacgtga tccggaaata tggagacccc |
| 121 | gggagcctct tcggcttctc gctggccatg cactggcaac tgcagcccga ggacaagcgg |
| 181 | ctgttgctcg tgggggcccc gcgcggagaa gcgcttccac tgcagagagc caacagaacg |
| 241 | ggagggctgt acagctgcga catcaccgcc cgggggccat gcacgcggat cgagtttgat |
| 301 | aacgatgctg accccacgtc agaaagcaag gaagatcagt ggatgggggt caccgtccag |
| 361 | agccaaggtc cagggggcaa ggtcgtgaca tgtgctcacc gatatgaaaa aaggcagcat |
| 421 | gttaatacga agcaggaatc ccgagacatc tttgggcggt gttatgtcct gagtcagaat |
| 481 | ctcaggattg aagacgatat ggatggggga gattggagct tttgtgatgg gcgattgaga |
| 541 | ggccatgaga aatttggctc ttgccagcaa ggtgtagcag ctacttttac taaagacttt |
| 601 | cattacattg tatttggagc cccgggtact tataactgga aagggattgt tcgtgtagag |
| 661 | caaaagaata acactttttt tgacatgaac atctttgaag atgggcctta tgaagttggt |
| 721 | ggagagactg agcatgatga aagtctcgtt cctgttcctg ctaacagtta cttaggtttt |
| 781 | tctttggact cagggaaagg tattgtttct aaagatgaga tcacttttgt atctggtgct |
| 841 | cccagagcca atcacagtgg agccgtggtt ttgctgaaga gagacatgaa gtctgcacat |
| 901 | ctcctccctg agcacatatt cgatggagaa ggtctggcct cttcatttgg ctatgatgtg |
| 961 | gcggtggtgg acctcaacaa ggatgggtgg caagatatag ttattggagc cccacagtat |
| 1021 | tttgatagag atggagaagt tggaggtgca gtgtatgtct acatgaacca gcaaggcaga |
| 1081 | tggaataatg tgaagccaat tcgtcttaat ggaaccaaag attctatgtt tggcattgca |
| 1141 | gtaaaaaata ttggagatat taatcaagat ggctacccag atattgcagt tggagctccg |
| 1201 | tatgatgact tgggaaaggt ttttatctat catggatctg caaatggaat aaataccaaa |
| 1261 | ccaacacagg ttctcaaggg tatatcacct tattttggat attcaattgc tggaaacatg |
| 1321 | gaccttgatc gaaattccta ccctgatgtt gctgttggtt ccctctcaga ttcagtaact |
| 1381 | attttcagat cccggcctgt gattaatatt cagaaaacca tcacagtaac tcctaacaga |
| 1441 | attgacctcc gccagaaaac agcgtgtggg gcgcctagtg ggatatgcct ccaggttaaa |
| 1501 | tcctgttttg aatatactgc taaccccgct ggttataatc cttcaatatc aattgtgggc |
| 1561 | acacttgaag ctgaaaaaga aagaagaaaa tctgggctat cctcaagagt tcagtttcga |
| 1621 | aaccaaggtt ctgagcccaa atatactcaa gaactaactc tgaagaggca gaaacagaaa |
| 1681 | gtgtgcatgg aggaaaccct gtggctacag gataatatca gagataaact gcgtcccatt |
| 1741 | cccataactg cctcagtgga gatccaagag ccaagctctc gtaggcgagt gaattcactt |
| 1801 | ccagaagttc ttccaattct gaattcagat gaacccaaga cagctcatat tgatgttcac |
| 1861 | ttcttaaaag agggatgtgg agacgacaat gtatgtaaca gcaaccttaa actagaatat |
| 1921 | aaattttgca cccgagaagg aaatcaagac aaattttctt atttaccaat tcaaaaaggt |
| 1981 | gtaccagaac tagttctaaa agatcagaag gatattgctt tagaaataac agtgacaaac |
| 2041 | agcccttcca acccaaggaa tcccacaaaa gatggcgatg acgcccatga ggctaaactg |
| 2101 | attgcaacgt ttccagacac tttaacctat tctgcatata gagaactgag ggctttccct |
| 2161 | gagaaacagt tgagttgtgt tgccaaccag aatggctcgc aagctgactg tgagctcgga |
| 2221 | aatcctttta aaagaaattc aaatgtcact ttttatttgg ttttaagtac aactgaagtc |
| 2281 | acctttgaca ccccatatct ggatattaat ctgaagttag aaacaacaag caatcaagat |
| 2341 | aatttggctc caattacagc taaagcaaaa gtggttattg aactgctttt atcggtctcg |
| 2401 | ggagttgcta aaccttccca ggtgtatttt ggaggtacag ttgttggcga gcaagctatg |
| 2461 | aaatctgaag atgaagtggg aagtttaata gagtatgaat tcagggtaat aaacttaggt |
| 2521 | aaacctctta caaacctcgg cacagcaacc ttgaacattc agtggccaaa agaaattagc |
| 2581 | aatgggaaat ggttgcttta tttggtgaaa gtagaatcca aaggattgga aaaggtaact |
| 2641 | tgtgagccac aaaaggagat aaactccctg aacctaacgg agtctcacaa ctcaagaaag |
| 2701 | aaacgggaaa ttactgaaaa acagatagat gataacagaa aattttcttt atttgctgaa |
| 2761 | agaaaatacc agactcttaa ctgtagcgtg aacgtgaact gtgtgaacat cagatgcccg |
| 2821 | ctgcgggggc tggacagcaa ggcgtctctt attttgcgct cgaggttatg gaacagcaca |
| 2881 | tttctagagg aatattccaa actgaactac ttggacattc tcatgcgagc cttcattgat |
| 2941 | gtgactgctg ctgccgaaaa tatcaggctg ccaaatgcag gcactcaggt tcgagtgact |
| 3001 | gtgtttccct caaagactgt agctcagtat tcgggagtac cttggtggat catcctagtg |
| 3061 | gctattctcg ctgggatctt gatgcttgct ttattagtgt ttatactatg gaagtgtggt |
| 3121 | ttcttcaaga gaaataagaa agatcattat gatgccacat atcacaaggc tgagatccat |
| 3181 | gctcagccat ctgataaaga gaggcttact tctgatgcat ag |

Antisense and RNAI sequence are derivable from these sequences. Antisense oligonucleotides are commonly from 12 to 50 bases in length, more preferably 15-30 bases length. Effective regions for targeting of antisense sequences may be found throughout the target nucleic acid. A preferred intragenic site is the region encompassing the translation initiation or termination codon of the open reading frame (ORF) of the gene. Since, as is known in the art, the translation initiation codon is typically 5′-AUG (in transcribed mRNA molecules; 5′-ATG in the corresponding DNA molecule), the translation initiation codon is also referred to as the ‘AUG codon,’ the ‘start codon’ or the ‘AUG start codon’. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms ‘translation initiation codon’ and ‘start codon’ can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, ‘start codon’ and ‘translation initiation codon’ refer to the codon or codons that are used in vivo to initiate translation of an mRNA molecule transcribed from a gene encoding Integrin beta 4, regardless of the sequence(s) of such codons.

It is also known in the art that a translation termination codon (or ‘stop codon’) of a gene may have one of three sequences, i.e., 5′-UAA, 5′-UAG and 5′-UGA (the corresponding DNA sequences are 5′-TAA, 5′-TAG and 5′-TGA, respectively). The terms ‘start codon region’ and ‘translation initiation codon region’ refer to a portion of such an MRNA or gene that encompasses from about 25 to about 50 contiguous nucleotides in either direction (i.e., 5′ or 3′) from a translation initiation codon. Similarly, the terms ‘stop codon region’ and ‘translation termination codon region’ refer to a portion of such an MRNA or gene that encompasses from about 25 to about 50 contiguous nucleotides in either direction (i.e., 5′ or 3′) from a translation termination codon.

The open reading frame (ORF) or ‘coding region,’ which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

RNAi molecules are similarly selected based on the sequence and defined parameters known for the selection of appropriate sequences. RNAi molecules may be single or double stranded, and generally have a length of 19 to 23 bases, although longer and shorter species can be used. A specific RNAi species useful in the method of the invention is based on the mouse sequence of beta4 cDNA (Genebank Acc. # L04678): nucleotides 113 to 131, counting from the A of the ATG translational start site, having the sequence GAGCTGTACCGAGTGCATC (Seq. ID. No. 4). This molecule, and the corresponding molecule based on the human sequence, and their use form a further aspect of this invention.

In one embodiment of the invention, the therapeutic agent is in combination with other therapy directed toward suppressing the activity of RPTKs known to cooperate with a6b4, including but not limited to ErbB2 (Her2), EGF-R, Met, and Ron. Specific examples of such inhibitors include the Her2 inhibitor trastuzumab (Herceptin™), and PPAR gamma ligands as described in U.S. Pat. No. 6,291,496, which is incorporated herein by reference

The invention and the evidence that established the efficacy and utility of the invention will now be further described with reference to the following non-limiting examples.

### EXAMPLE 1

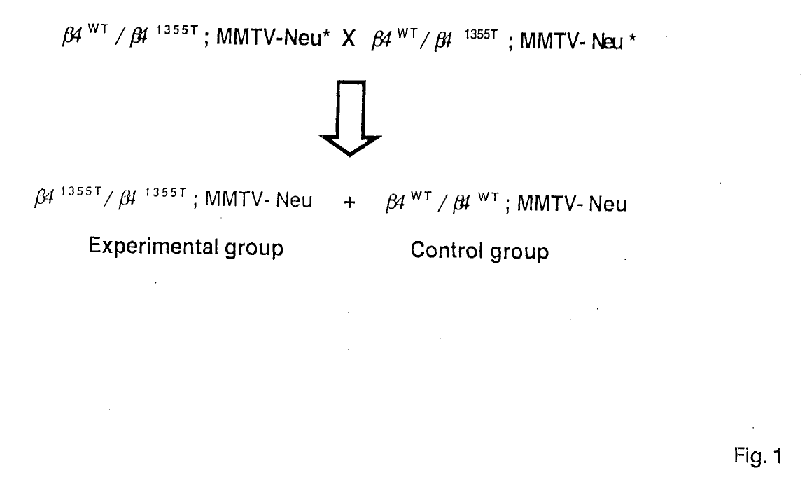

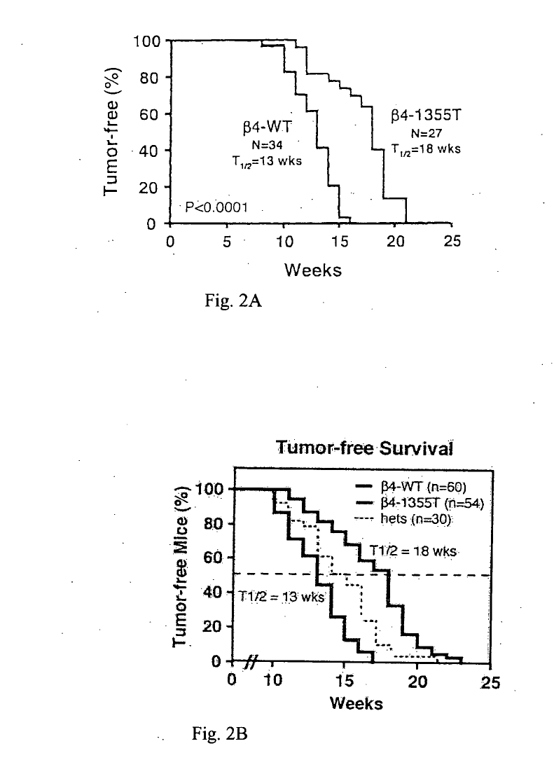

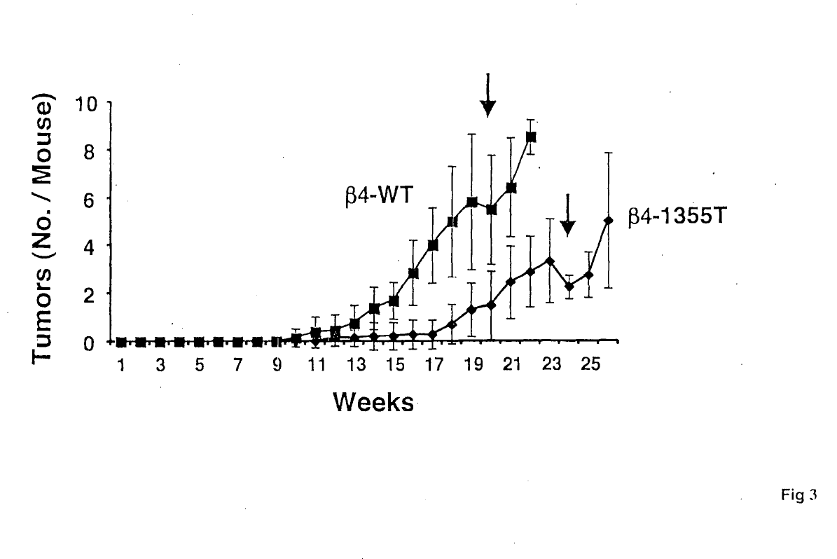

The MMTV-Neu Nd-YD transgene was introduced into both wild-type and b4-1355T mice of FVB background using the breeding strategy outlined in FIG. 1 (asterick points to Neu mutant. Tumor onset was evaluated by palpation, and mice carrying palpable mammary nodules considered affect. As shown in FIG. 2A, the b4 mutant mice lived free of tumors significantly longer than the corresponding control mice. In addition, the b4 mutant mice developed, on average, a smaller number of individual tumors in their mammary glands. (FIG. 3). In a second set of experiments, the test was repeated with a larger number of mice in each group. As reflected in FIG. 2B, the same t_{1/2} was observed. The dotted line in FIG. 2B corresponds to heterozygous MMTV-Neu(YD); b4^{+/1355T} mice. These results indicate that a6b4 signaling promotes tumorigenesis in this model of breast cancer.

### EXAMPLE 2

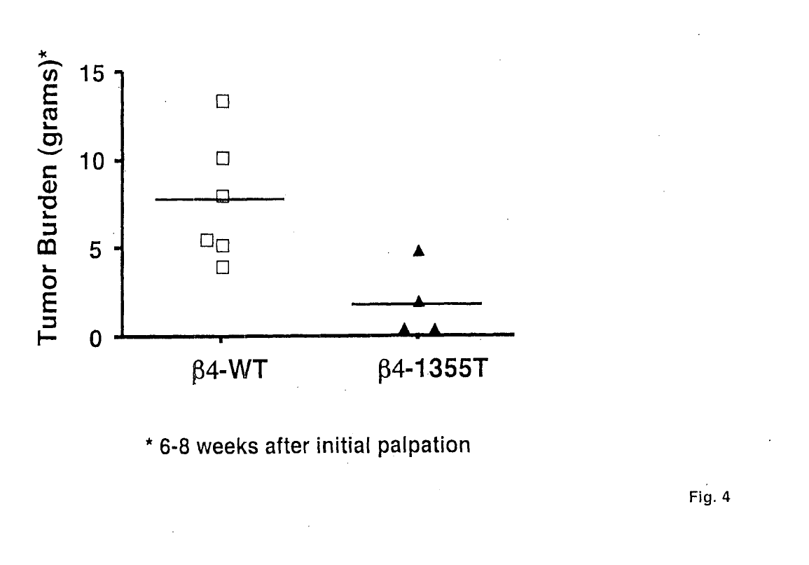

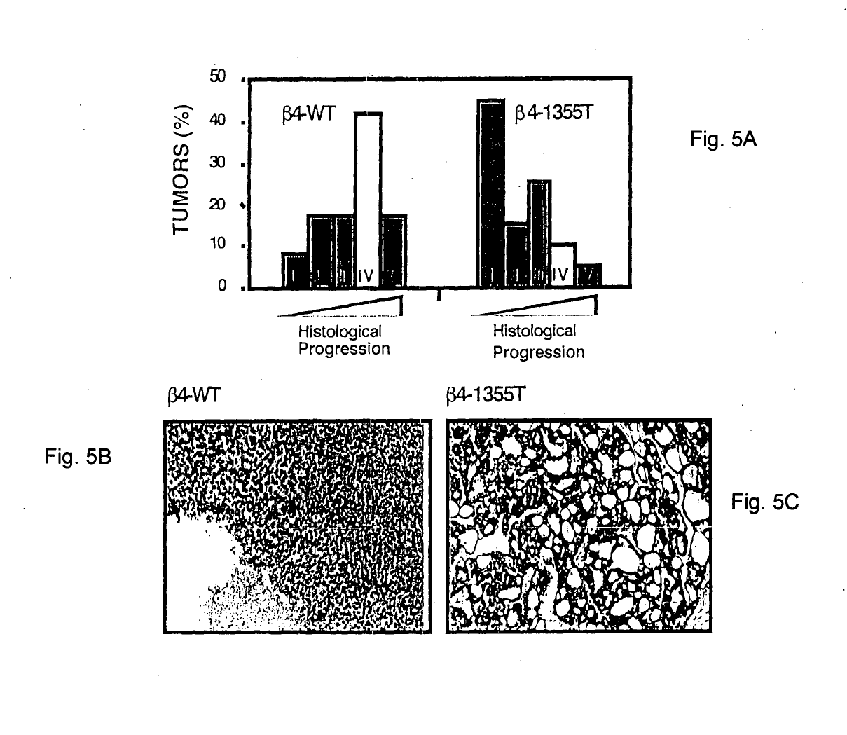

Tumor growth was evaluated at 6 to 8 weeks after initial detection of tumors. As shown in FIG. 4, the mmary carcinomas of b4 mutant mice grew at a slower rate than those of the control mice, indicating that a6b4 signaling promotes tumor growth. Histological analysis indicated that tumors arising in the b4 mutants background were significantly more differentiated (mostly adenocarcinomas) that those arising in the the wild-type background (mostly undifferentiated invasive carcinomas). (FIGS. 5A-C) Furthermore, immunohistochemistry showed that the b4 mutant retained an apparently intact laminin-containing basement membrane, whereas the wildtype had disrupted the basement membrane and progressed to a frankly invasive stage.

### EXAMPLE 3

To begin to examine the mechanism by which a6b4 signaling promotes tumor progression, we isolated mammary tumor cell lines from both wild-type and b4 mutant mice. Upon plating on a 2-D matrix, control and b4 mutant tumor cells grew at similar rates. However, when suspended in a Matrigel (a 3-D gel containing basement membrane components) the wild-type tumors proliferated rapidly, producing disorganized aggregates. In contrast, the b4 mutant cells gave rise to small cystic structures resembling normal mammary acini. Taken together, these results provide, for the first time, genetic evidence that a4b6 signaling accelerates breast cancer progression by promoting the transition from adenocarcinoma in situ to invasive and metastatic carcinoma and by promoting tumor growth.

### EXAMPLE 4

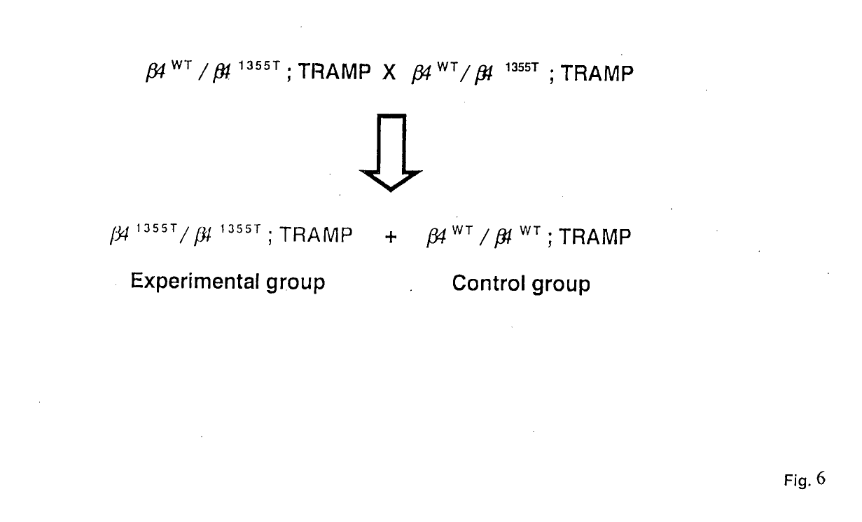

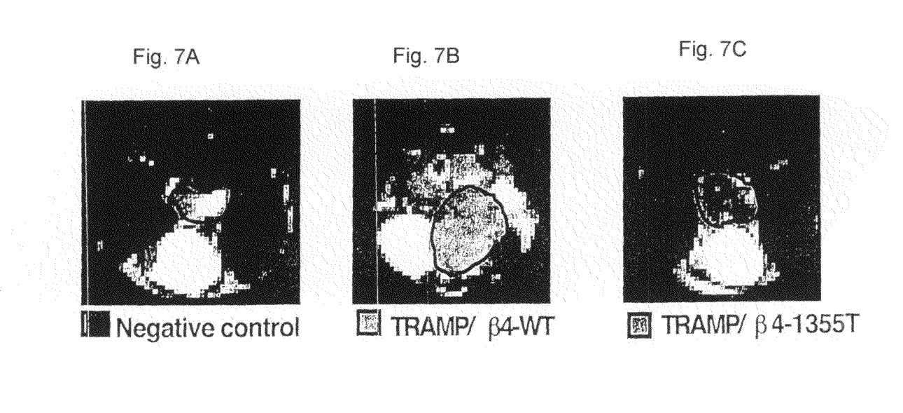

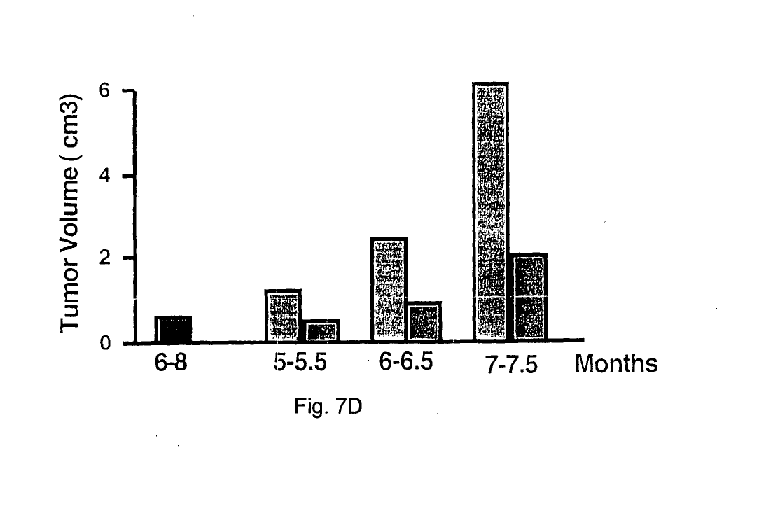

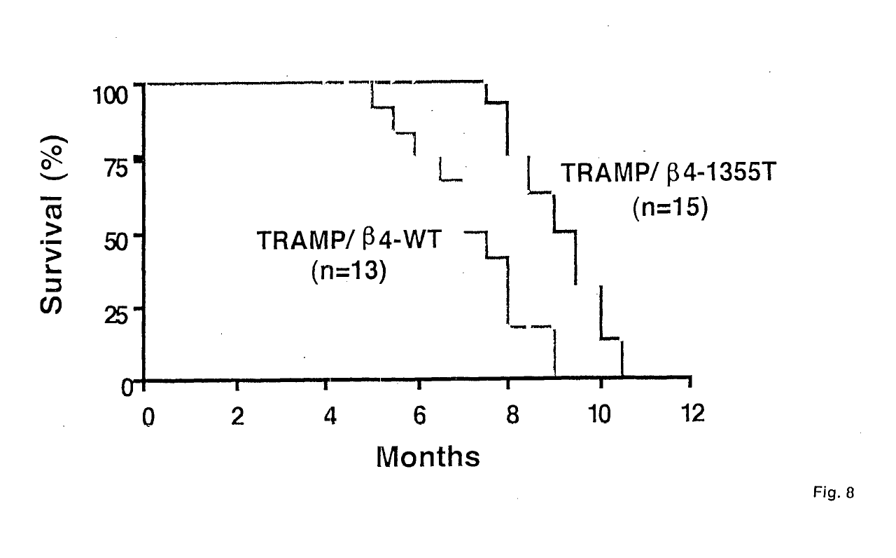

Transgenic mice expressing an SV-40-Tag oncogene from the prostate-specific promoter of Probasin (TRAMP mice) develop prostate cancer with complete pentrance (Gingrich et al., 1992). To example the role of a6b4 signaling in prostate carcinoma progression, we introduced the Probasin-SV-40-Tag trfansgene in both wild-type and b4-mutant mice following the breeding strategy outlined in FIG. 6. MRI analysis indicated that tumor onset and growth were delaying in mice carrying the b4 mutation as compared to mice expressing mild-type b4. (FIG. 7A-D). In addition, the overall survival of the b4 mutant TRAMP mice was observed to be longer than that of the b4-wild-type TRAMP mice. (FIG. 8).

### EXAMPLE 5

Consistent with the results in mammary tumors, histological analysis of prostate tumors from b4-mutant and b4-wild-type TRAMP mice indicated that the tumors arising in the b4-mutant background were considerably more differentiated than those arising in the b4- wild-type background. Anti-Ki67 staining, which marks the nuclei of proliferating cells, revealed that b4-mutant tumors have a significantly reduced prolliferative index as compared to the b4-wild-type tumors. Furthermore, b4 was polarized in correspondence of the basement membrane in the tumors of b4-mutant but not b4-wild-type background. Taken together, these findings demonstrate that in the prostate, as in the mammary gland, a6b4 signaling promotes the transition from well differentiated adenocarcinoma with intact basement membrane to invasive carcinoma.

### EXAMPLE 6

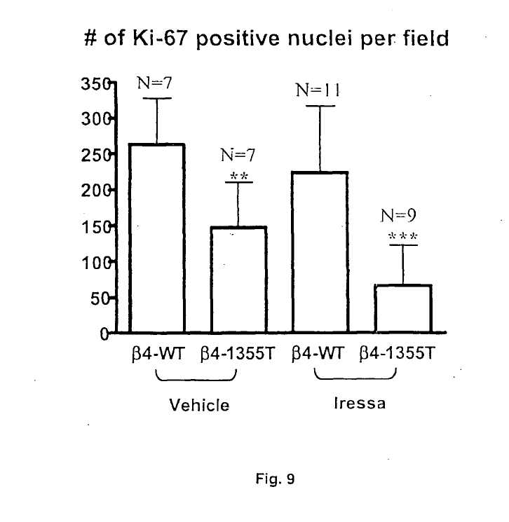

We have treated with the kinase inhibitor IRESSA, which inhibits both the EGF-R and Neu (de Bono and Rowinsky, 2002), wild-type and b4-mutant mice carrying MMTV-Neu tumors. The results indicated that IRESSA causes a much larger inhibition of tumor cell proliferation in b4-mutant mice than it does in wild-type mice (FIG. 9). About 80% of the IRESSA-treated tumors arising in b4-mutant mice regress, compared to about only 20% in the control group (FIG. 2). This striking result indicates that blockage of a6b4 increases the effectiveness of cancer therapy with RTK inhibitors, a group which includes all tumor types expressing a6b4 and carrying amplified or activated versions of Neu, EGF-R, and Met RTKs (Bacus et al., 1994; Longati et al., 2001; Sawyers, 2002).

MMTV-Neu (YD) mice bearing mammary tumors (>0.5 cm in diameter) were treated with Iressa (100 mg/Kg/day) or vehicle (0.1% Tween-80) by gastric gavage for 1 month or 7 days. Tumor sections were stained with anti-Ki-67 Mab, which labels proliferating cells. There is no significant difference in Ki-67 staining between mice treated for 1 month or 7 days. Unpaired, two-tailed t-test showed: P<0.01 between WT-vehicle and 1355T-vehicle; P<0.001 between WT-Iressa and 1355T-Iressa; P=0.016 between 1355T-vehicle and 1355T-Iressa (FIG. 9)

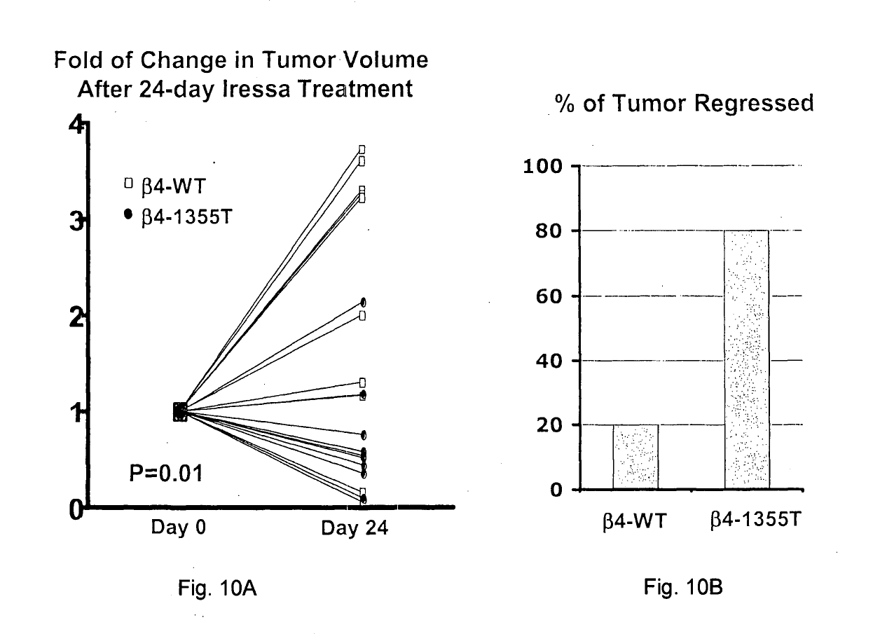

MMTV-Neu (YD) mice bearing mammary tumors (>0.5 cm in diameter) were treated with Iressa (100 mg/Kg/day) by gastric gavage for 24 days. Tumor volumes were measured by caliper. FIG. 10A shows the fold changes in tumor volume from day 0 to day 24, with each line representing one mouse (N=10 for each group, P<0.01). FIG. 10B shows the percentage of mice with regressed tumors in each group.

### EXAMPLE 7

In the normal mammary gland, basal myoepithelial cells express significant levels of a6b4, whereas luminal epithelial cells express lower amounts of this integrin. The neoplastic cells of MIN lesions in Neu(YD)/b4-WT mice did not express the myoepithelial marker smooth muscle alpha-actin but exhibited significantly elevated levels of b4 as compared to normal luminal cells. b4 was no longer concentrated at the basement membrane junction but was instead diffusely distributed over the cell surface. In contrast, the levels of its matrix ligand, laminin-5, in the basement membrane were severely reduced. As MIN lesions progressed to invasive carcinomas, laminin-5 became undetectable, but the levels of b4 remained elevated. The tumors in Neu(YD)/b4-1355T mice exhibited a similar up-regulation of b4 and down-regulation of laminin-5. However, the mutant b4 integrin remained in part concentrated at the basement membrane junction in MIN lesions of these mice, indicating that b4 signaling may contribute to disruption of epithelial polarity. Since individual tumors of Neu(YD)/b4-1355T mice grew only at a modestly reduced rate (approximately 70% of control value) and contained a number of microvessels similar to that of control Neu(YD)/b4-WT mice, we examined if loss of b4 signaling inhibits mammary tumor induction or initial growth. Histological analysis revealed that Neu(YD)/b4-1355T mice exhibit a severely diminished number of mammary intraepithelial neoplasia (MIN) lesions at 13 weeks of age (2.1±2.4 per median longitudinal section, n=7 mice) as compared to Neu(YD)/b4-WT mice (10.1±6.6, n=8 mice; P=0.01), indicating that loss of b4 signaling inhibits mammary tumor onset and initial growth.

To dissect the mechanism by which loss of b4 signaling suppresses mammary tumorigenesis, we examined preneoplastic and MIN lesions. Anti-Ki-67 staining showed that activated ErbB2 induces robust epithelial cell proliferation prior to overt morphological transformation in the ducts and lobules of Neu(YD)/b4-WT mice. (FIG. 11A) Mammary glands from age-matched wild-type mice contained only scattered Ki-67-positive cells. By contrast, cell proliferation was only modestly increased in the ducts and lobules of Neu(YD)/b4-1355T mice (FIG. 11A). Furthermore, MIN lesions from Neu(YD)/b4-WT mice contained a significantly higher proportion of proliferating tumor cells (approximately 40%) than those from Neu(YD)/b4b-1355T mice (approximately 20%) (FIG. 11B). Staining with antibodies to cleaved caspase-3 indicated modest apoptotic rates in most MIN lesions from Neu(YD)/b4-WT mice. However, a subset of early MIN lesions with a pervious lumen in Neu(YD)/b4-1355T mice contained a significant number of apoptotic cells (8.9±4.4%, n=4). Interestingly, these lesions had a high proliferative index, suggesting that aberrant cell proliferation contributes to apoptosis in these lesions. By contrast, early MIN lesions with a similarly high proliferative rate in Neu(YD)/b4-WT mice exhibited only modest apoptosis (1.8±0.7%, n=4, P=0.02). These results indicate that b4 signaling promotes cell proliferation throughout the pre-neoplastic and the MIN stage and suppresses oncogene-induced apoptosis prior to luminal filling.

### EXAMPLE 8

ErbB2 mammary tumors progress from MIN to invasive carcinoma through steps characterized by increasing degrees of de-differentiation. At 5 months of age, Neu(YD)/b4-1355T mice exhibited a high proportion of well and moderately differentiated tumors characterized by a glandular appearance. By contrast, Neu(YD)/b4-WT mice had developed predominantly poorly differentiated tumors (FIG. 12), further indicating that deletion of the b4 signaling domain inhibits histological progression. As they progress, mammary tumors of MMTV-Neu(YD) mice retain expression of E-cadherin but lose the tight junction component Zonula Occludens 1 (ZO-1) like their human counterpart. To further examine the effect of b4 signaling on tumor progression, we studied the expression of these adhesion components in moderately differentiated tumors from Neu(YD)/b4-WT and Neu(YD)/b4-1355T mice. Both types of tumors retained expression of E-cadherin at the cell surface. However, tumors from Neu(YD)/b4-WT mice exhibited severely decreased ZO-1 staining. Tight junction strands were not apparent, and most of the ZO-1 reactivity was located ectopically in intracellular granules. By contrast, tumors from Neu(YD)/b4-1355T mice contained clear tight junction strands between adjacent cells. Within pseudo-glandular structures, these strands formed a continuous collar surrounding the apical pole of cells. These observations indicate that loss of b4 signaling opposes disassembly of tight junctions and internalization of their components.

### EXAMPLE 9

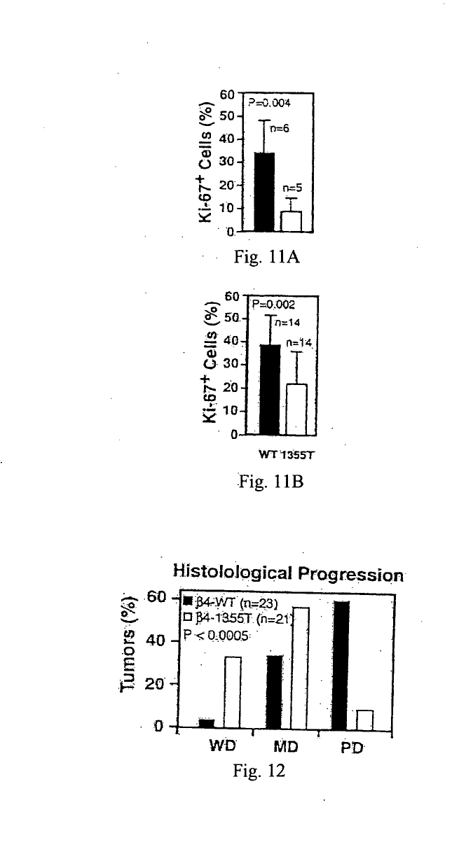

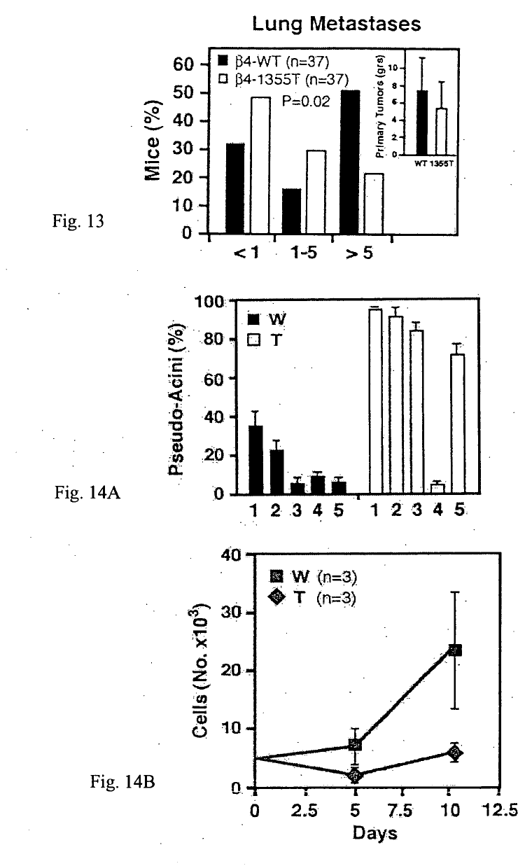

Since mammary tumors of MMTV-Neu(YD) mice primarily metastasize to the lung, we examined the lungs of both types of mice 7.5 weeks after primary tumor onset. We found that most of the Neu(YD)/b4-WT mice had developed a large number of metastases, but most of the Neu(YD)/b4-1355T mice exhibited either no metastases or only a few of them (FIG. 13). Cumulative primary tumor burden was similar in the two types of mice (FIG. 13, inset). This result indicates that loss of b4 signaling inhibits spontaneous metastasis to the lung.

### EXAMPLE 10

Tight junctions have emerged as key regulators of mammalian epithelial polarity and adhesion. Prompted by the observation that deletion of the b4 signaling domain increases the organization of tight junctions in mammary tumors, we used an ex vivo approach to examine the effect of b4 signaling on epithelial adhesion and polarity. Primary ErbB2-transformed cells isolated from Neu(YD)/b4-WT and Neu(YD)/b4- 1355T mice were found to express similar levels of ErbB2. However, tumor cells expressing wild-type b4 exhibited a spindle-like morphology and their margins tended to overlap, whereas those expressing b4-355T had polygonal shapes and their margins appeared closely apposed. Anti-ZO-1 staining revealed that cells expressing wild-type b4 had severely disrupted tight junctions. Anti-E-cadherin and anti-beta-catenin staining showed that they also had disorganized adherens junctions. Strikingly, tumor cells expressing b4-1355T exhibited well-organized adherens and tight junctions, indicating that loss of b4 signaling restores epithelial adhesion in ErbB2-transformed MECs. Gefitinib (Iressa), which was developed to block the EGFR but also inhibits activated ErbB2 with an IC50 of approximately 1 μM, promoted reassembly of both adherens and tight junctions in tumor cells expressing wild-type b4. Since ErbB2-transformed mouse mammary epithelial cells do not express detectable levels of the EGF-R in vitro, it is likely that Iressa exerts its effect in these cells by inhibiting ErbB2. These results suggest that the b4 integrin cooperates with ErbB2 to induce disruption of epithelial adhesion.

In 3D Matrigel, normal MECs form monolayered acinar structures and undergo growth arrest. By contrast, tumorigenic MECs form disorganized solid aggregates and continue to proliferate. Consistent with their neoplastic nature, tumor cells expressing wild-type b4 formed expansive solid spheroids in 3D Matrigel. Immunofluorecent staining showed that these structures were profoundly disorganized. Laminin-5 was deposited both around and inside tumor cell aggregates and the b4 integrin was diffusely distributed over the cell surface. In addition, E-cadherin and beta-catenin were not concentrated at adherens junctions and ZO-1 was present in intracellular granules. In striking contrast, most tumor cells expressing b4-1355T assembled pseudo-acinar structures. These structures possessed a lumen and exhibited a distinctive epithelial organization and polarity. Moreover, the mutant integrin was concentrated at the basal cell surface and laminin-5 was deposited exclusively underneath this surface. E-cadherin and beta-catenin were partially localized at cell-to-cell junctions and ZO-1 was concentrated in tight junction strands at the apical junctional complex. These findings indicate that loss of b4 signaling restores a significant degree of epithelial polarity to ErbB2-transformed MECs.

To examine the effect of b4 signaling on growth control, we monitored tumor growth in 3D Matrigel. Over an 11-day period, the disorganized aggregates formed by tumor cells expressing wild-type b4 expanded continuously. By contrast, the pseudoacinar spheroids formed by cells expressing mutant b4 underwent a very limited expansion (FIGS. 14A and B), indicating that 4 signaling is required for ErbB2-induced epithelial overproliferation in 3D Matrigel. Together, these findings suggest that the b4 integrin cooperates with ErbB2 to coordinately disrupt epithelial organization and growth control.

### EXAMPLE 11

The analysis of tumor progression in genetically engineered mice is complicated by the accumulation of secondary mutations. Because b4 signaling promotes tumor cell proliferation at the MIN stage, it may favor the accumulation of such mutations. To examine if the b4 integrin disrupts epithelial adhesion by a direct signaling mechanism, we generated isogenic Neu-transformed mammary tumor cells expressing either wildtype or mutant b4 by using an RNAi-reconstitution strategy. To construct retroviral vectors encoding human b4-WT or b4-13355T in combination with a shRNA to mouse b4, we first ligated an oligonucleotide encoding a shRNA targeting the mouse b4 sequence 5′GAGCTGTACCGAGTGCATC3′ (SEQ ID No.: 5) into the BgIII and BamHI sites of LTRH1. We then replaced the CD4 coding sequence in LTRHH1 with an IRES-hygromycin resistance cassette. Finally, we subcloned human b4-WT or b-1355T cDNAs into the EcoRI site immediately upstream of this cassette. The resulting vectors were named LTRH1-b4-WT and LTRH1-b4-1355T. To construct a retroviral vector encoding TAM67, the TAM67 cDNA was subcloned in pBMN-IRES-EGFP (from Gary Nolan, Stanford University). The Stat3-beta vector was constructed by subcloning the Stat3-beta cDNA into pMSCV-IRES-EGFP and provided by Jackie Bromberg (Department of Medicine, MSKCC). pLVTH lentiviral vectors encoding a control shRNA containing a scrambled anti-ALK sequence and a shRNA targeting STAT3 were previously described (Chatterjee et al., 2004). The pLKO1 vector encoding a shRNA targeting c-Jun (5′-CCGGGAAGCGCAT GAGGAACCGCATCTCGAGATGCGGTTCCTCATGCGCTTCTTTTTG-3′) Seq ID. No: 6 was obtained from Open Biosystems. It incorporates an iRNA sequence that mediates efficient and specific knock down of c-Jun.

The Neu-b4-1355T cells assembled well-organized adherens and tight junctions in culture. By contrast, the Neu-b4-WT cells failed to organize both types of junctions, unless they were treated with Iressa. These results indicate that the b4 integrin induces disruption of cell junctions by a direct signaling mechanism.

### EXAMPLE 12

Constitutively active RTKs and SFKs can induce disassembly of adherens junctions through SNAIL/Slug-mediated repression of E-cadherin or tyrosine phosphorylation and endocytosis of the E-cadherin/beta-catenin complex. Studies with inhibitors suggested that SFK signaling contributes to disruption of epithelial adhesion in Neu-b4-WT cells, but PI-3K and MMP 1, 2, 3, and 9 do not. We did not, however, detect reduced expression or tyrosine phosphorylation of E-cadherin in Neu-b4-WT cells. In addition, beta-catenin was phosphorylated on tyrosine to similar levels in Neu-b4-WT and Neu-b4-1355T cells. These experiments indicate that b4 enables ErbB2 to disrupt epithelial adhesion through a novel mechanism.

To examine the effect of b4 signaling on mammary tumor architecture in its physiological context, we implanted Neu-b4-WT and Neu-b4-1355T cells in the mammary fat pad of athymic nude mice. Over a 3-week period, the Neu-b4-WT cells formed tumors approximately two-fold larger than those generated by Neu-b4-1355T cells.

The tumors expressing wild-type b4 had a solid histological appearance and lacked signs of tissue organization. Immunofluorescent staining detected scattered, short fibrils of laminin-5 and collagen IV. Beta-catenin was diffusely distributed near the cell surface but ZO-1 was predominantly present in intracellular vesicles. By contrast, the tumors expressing mutant b4 exhibited a striking pseudo-glandular organization.

The epithelial cells surrounding the lumens of glandular structures were supported by a continuous, albeit partially disorganized, basement membrane containing laminin-5 and collagen IV. They exhibited many seemingly normal beta-catenin-containing junctions and assembled ZO-1-containing tight junctions toward their apical pole.

These results provide evidence that b4 signaling exerts a direct effect on epithelial adhesion, polarity, and organization in vivo.

To examine if b4 signaling directly affects tumor cell proliferation and survival in vivo, we stained tumor sections with antibodies to Ki-67 and to cleaved caspase-3. The Neu-b4-1355T tumors contained significantly fewer proliferating cells as compared to Neu-b4-WT tumors. In addition, whereas Neu-b4-WT tumors had only scattered apoptotic cells, Neu-b4-1355T tumors contained a significant number of apoptotic cells. Notably, these cells were concentrated in the lumens of pseudo-glandular structures, indicating that b4 signaling suppresses anoikis. These results illustrate the ability of b4 signaling to coordinately disrupt epithelial polarity and growth control in the mammary gland.

### EXAMPLE 13

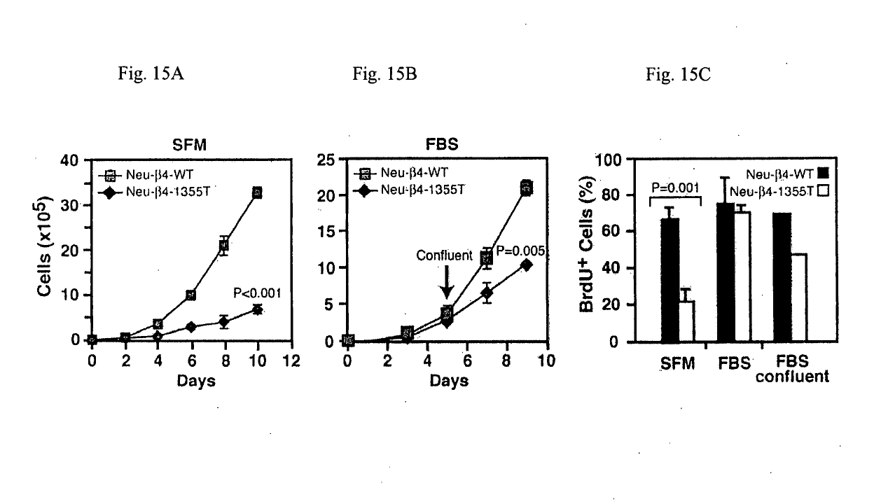

To further examine the effect of b4 signaling on ErbB2-mediated proliferation, we examined the ability of Neu-b4-WT and Neu-b4-1355T cells to proliferate in vitro. In the absence of serum, the Neu-b4-1355T cells proliferated at a dramatically reduced rate in comparison to Neu-b4-WT cells (FIGS. 15A-C). In the presence of serum, both types of cells proliferated at similar rates during the logarithmic phase of growth.

However, upon reaching confluency, the Neu-b4-1355T cells proliferated less rapidly as compared to control Neu-b4-WT cells. These results indicate that b4 signaling enables activated ErbB2 to promote growth factor-independent mitogenesis and contributes to a certain extent to its ability to disrupt contact inhibition.

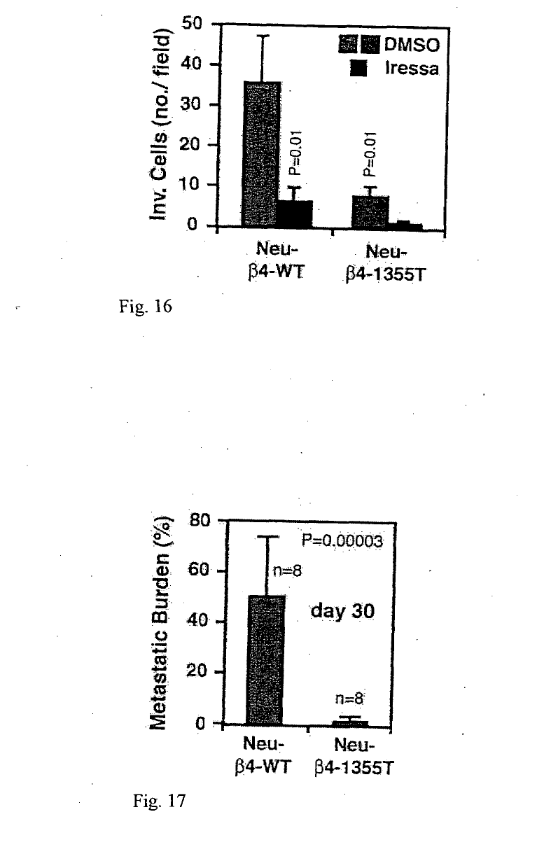

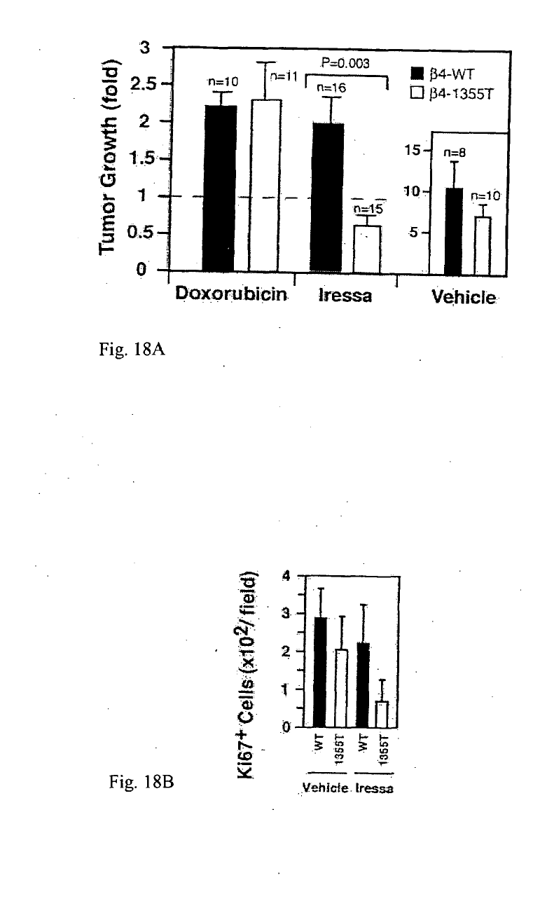

We next examined the effect of b4 signaling on mammary tumor cell invasion and metastasis. Consistent with their inability to form cell-to-cell junctions, Neu-b4-WT cells scattered extensively in culture. In contrast, the Neu-b4-1355T cells grew as clusters of tightly adhering cells (FIG. 4C). When subjected to Matrigel invasion assay, the Neu-b4-WT cells invaded efficiently and Iressa prevented their invasion. In contrast, the Neu-b4-1355T cells invaded poorly through Matrigel (FIG. 16). Finally, upon intravenous injection in athymic nude mice, Neu-b4-WT cells produced numerous, large metastases in the lung, but Neu-b4-1355T formed only a few micrometastases (FIG. 17). Together with the observation that Neu(YD-b4-1355T mice progress to lung metastasis less efficiently than Neu(YD)-b4-WT mice, these results indicate that b4 signaling promotes mammary tumor invasion and metastasis.

### EXAMPLE 14

Biochemical studies were performed to study the molecular mechanism by which b4 signaling promotes mammary tumorigenesis. Co-immunoprecipitation analysis revealed that ErbB2 forms a complex with a6b4 and induces tyrosine phosphorylation of b4. Formation of the complex and tyrosine phosphorylation of b4 did not require ligand binding to a6b4 or the kinase activity of ErbB2 or SFKs. Whereas Iressa suppressed tyrosine phosphorylation of both ErbB2 and b4, PP2 suppressed phosphorylation of b4 but only partially inhibited phosphorylation of ErbB2, suggesting that ErbB2 induces phosphorylation of b4 through activation of SFKs. Iressa suppressed phosphorylation of ErbB2 at its major autophosphorylation site, which mediates recruitment of Shc and Grb2, but did not affect activation of SFKs. In contrast, PP2 exerted the opposite effects. These results indicate that ErbB2 forms a complex with a6b4 and suggest that ErbB2 induces phosphorylation of b4 through SFKs.

We next studied the effect of deletion of the b4 signaling domain on the assembly and function of the b4-ErbB2 complex. Co-immunoprecipitation analysis showed that deletion of the b4 signaling domain uncouples a6b4 from ErbB2 and inhibits activation of integrin-associated SFKs. In addition, it reduces the amount of SFKs associated with ErbB2. These results indicate that the b4 signaling domain is required for assembly of the b4-ErbB2 complex and promotes SFK association with ErbB2.

Since SFKs can phosphorylate Y845 in the P-loop of the EGF-R, we examined the effect of SFK inhibition on phosphorylation of the corresponding tyrosine in the P-loop of ErbB2. Immunoblotting of total lysates showed that PP2 and Iressa inhibit phosphorylation of the P-loop of ErbB2 to a significant extent and, when used in combination, completely suppress phosphorylation of this site . Similar results were obtained upon replacing PP2 with Dasatinib, which inhibits SFKs at nanomolar concentrations. Notably, deletion of the b4 signaling domain suppressed phosphorylation of the P-loop but not the major autophosphorylation site of ErbB2. These results indicate that the 4 signaling domain contributes to phosphorylation of the P-loop of ErbB2 by promoting SFK association with the RTK. Thus, b4 functions both upstream and downstream of ErbB2 and deletion of the b4 signaling domain uncouples b4 from ErbB2, suppressing joint signaling.

### EXAMPLE 15

ErbB2 activates Ras, PI-3K, and JAK-STAT signaling . To examine the effect of b4 on ErbB2 signaling, we compared the levels of activation of ERK, JNK, and Akt in Neu-b-WT and Neu-b4-1355T cells stably adhering to laminin-5 or collagen I, as a control. These kinases were activated to similar levels in both types of cells on either substrate. Since b4 signaling controls translocation of activated MAP kinases to the nucleus in EGF-treated keratinocytes, we next monitored nuclear accumulation of activated ERK and JNK in Neu-b4-WT and Neu-b4-1355T cells. Using immunofluorescent staining, we did not detect significant differences in nuclear accumulation of activated ERK between the two types of cells. However, we found PJNK concentrated in the nucleus in Neu-b4-WT cells but diffusely distributed in the cytoplasm in Neu-b4-1355T cells, suggesting that b4 signaling controls nuclear accumulation of activated JNK. In agreement with this conclusion, phosphorylation of c-Jun at its JNK phosphorylation site, S63, was substantially suppressed in Neu-b4-1355T cells plated on either laminin-5 or collagen I. Iressa inhibited phosphorylation of c-Jun in Neu-b4-WT cells, but at concentrations higher than those required to suppress activation of ERK, suggesting that b4 signaling sustains ErbB2-dependent activation of c-Jun. Biochemical fractionation and imaging studies have demonstrated that a6b4 and b1 integrins control nuclear translocation of MAP kinases. Our results are consistent with these findings and indicate that b4 contributes to ErbB2 signaling by promoting nuclear translocation of JNK and therefore phosphorylation of c-Jun.

Prior studies have linked SFK-mediated phosphorylation of the P-loop of ErbB receptors to JAK-STAT signaling. Since deletion of the b4 signaling domain reduces SFK activation and association with ErbB2 and decreases phosphorylation of the P-loop of ErbB-2, we examined the effect of this deletion on STAT3 activation. Phosphorylation at Y705 is essential for dimerization and activation of STAT3. We found STAT3 constitutively phosphorylated at Y705 in Neu-b4-WT cells plated on laminin-5 or collagen I. In contrast, phosphorylation of this residue was almost undetectable in Neu-b4-1355T cells on either matrix ligand. As observed for c-Jun, Iressa inhibited phosphorylation of STAT3 in Neu-b4-WT cells at concentrations higher than those necessary to suppress activation of ERK, suggesting that b4 signaling sustains ErbB2-mediated activation of STAT3. STAT3 S727, which is targeted by ERK, was phosphorylated at similar levels in both types of cells. Together, these results indicate that deletion of the b4 signaling domain impairs ErbB2-mediated activation of STAT3.

To examine if the b4-ErbB2 complex induces phosphorylation of c-Jun and activates STAT3 in vivo, we stained sections of mammary glands from 13-week old Neu(YD)/b4-WT and Neu(YD)/b4-1355T mice with anti-P-c-Jun and anti-PY-STAT3 antibodies. Most pre-neoplastic cells and tumor cells in MIN lesions of Neu(YD)/b4-WT mice exhibited prominent nuclear staining for activated c-Jun and STAT3. In contrast, most epithelial cells in similar lesions of Neu(YD)/b4-1355T mice displayed either weak staining or no staining, and only a few cells exhibited strong nuclear staining . Immunoblotting on mammary fat pad lysates confirmed the reduction of c-Jun phosphorylation in the lesions of Neu(YD)/b4-1355T mice. Immunoblotting with anti-PY-STAT3 yielded inconclusive results, presumably because of the prominent activation of STAT3 in stromal cells. Together, these results indicate that the b4 integrin enables ErbB2 to activate c-Jun and STAT3.

### EXAMPLE 16

JNK-mediated phosphorylation of c-Jun contributes to transcriptional activation and to oncogenesis. To examine the role of c-Jun during ErbB2- driven mammary tumorigenesis, we used a dominant negative form of c-Jun (TAM67), which has been shown to suppress breast cancer cell proliferation by specifically interfering with AP-1-dependent transcription. Neu-b4-WT cells were transduced with a retroviral vector encoding TAM67 or GFP, as a control. Expression of TAM67 inhibited mammary tumor cell proliferation in vitro and tumorigenicity in vivo. This mutant, however, did not restore assembly of tight or adherens junctions in Neu-b4-WT cells, and it did not suppress the ability of these cells to invade through Matrigel in vitro. These results suggest that c-Jun is necessary for ErbB2-mediated hyperproliferation but not for disruption of epithelial adhesion.

STAT3 is frequently activated in human breast cancer samples. In addition, recent studies have indicated that STAT3 activation is sufficient to transform mammary epit helial cells in vitro. Expression of a dominant negative mutant form of STAT3 (STAT3-beta) did not inhibit mammary tumor cell proliferation in vitro and tumorigenicity in vivo. However, it restored assembly of tight junctions to a significant extent and formation of adherens junctions partially. In addition, STAT3-beta inhibited Matrigel invasion and experimental metastasis. These results suggest that STAT3 is necessary for ErbB2-mediated disruption of cell junctions and invasion but not for hyperproliferation.

To obtain additional evidence that the b4-ErbB2 complex promotes disruption of epithelial adhesion and hyperproliferation through activation of STAT3 and c-Jun, we used lentiviral vectors encoding shRNAs targeting each one of the two transcription factors. Neu-b4-WT cells transduced with each of these vectors exhibited significant knock down of the corresponding target protein. Knock down of c-Jun suppressed tumor cell hyperproliferation but did not restore assembly of tight junctions. By contrast, knock down of STAT3 partially restored assembly of tight junctions without affecting proliferative rates. Collectively, these data indicate that the b4-ErbB-2 complex promotes hyperproliferation through activation of c-Jun and it disrupts epithelial adhesion largely through activation of STAT3.

### EXAMPLE 17

As shown in Example 6, treatment with Iressa was more effective in Neu-b4-1355T mutant mice than in b4-WT mice. We further compared this change in effectiveness to the effectiveness of doxorubicin in the two types of mice. effectiveness. Iressa induced regression of Neu(YD)/b4-1355T tumors. In contrast, it only reduced the rate of growth of Neu(YD)/b4-WT tumors. Iressa suppressed activation of ErbB2 in both types of tumors, consistent with its equal apparent IC50 in Neu-b4-WT and Neu-b4-1355T cells in vitro. However, whereas Iressa inhibited tumor cell proliferation in Neu(YD)/b4-1355T mice, it exerted a more modest inhibitory effect in Neu(YD)/b4-WT mice. In addition, the drug increased tumor apoptosis in Neu(YD)/b4-1355T mice to a larger extent than it did in Neu(YD)/b4-WT mice, although overall apoptotic rates were in both cases very low (less than 1%).

To examine the specificity of the increased response of Neu(YD)/b4-1355T tumors to anti-ErbB2 therapy, we treated tumor-bearing Neu(YD)/b4-WT and Neu(YD)/b4-1355T mice with the chemotherapeutic drug Doxorubicin. Unlike Iressa, Doxorubicin reduced the growth rate of tumors in Neu(YD)/b4-WT and Neu(YD)/b4-1355T mice by a similar extent, and it failed to induce tumor regression in either type of mouse. These results indicate that b4 signaling specifically promotes resistance to anti-ErbB2 therapy.

### EXAMPLE 18

To examine the potential molecular basis of the differential effect of Iressa on tumors expressing wild type or mutant b4, we examined the activation of c-Jun, STAT3, ERK, and Akt in tumor lysates. c-Jun and STAT3 were activated in Neu(YD)/b4-WT tumors to levels higher than in Neu(YD)/b4-1355T tumors. Importantly, Iressa did not suppress but seemed to increase to a small extent activation of the two transcription factors in both types of tumors. It is possible that this apparent increase reflects the elimination of tumor cells exhibiting low levels of activated c-Jun and STAT3. Furthermore, Iressa inhibited activation of ERK and Akt in Neu(YD)/b4-1355T tumors by an extent larger than it did in Neu(YD)/b4-WT tumors.

In agreement with these observations, Iressa inhibited activation of ERK and Akt in Neu-b4-1355T cells in vitro at doses lower than in Neu-b4-WT cells. Together, these observations indicate that the b4 integrin sustains ErbB2-dependent mammary oncogenesis in vivo through activation of c-Jun and STAT3 and enhancement of signaling to ERK and Akt.

### REFERENCES

The following references are cited herein, and are incorporated herein by reference in their entirety.

Abdel-Ghany M, Cheng H C, Elble R C, Pauli B U, The breast cancer beta 4 integrin and endothelial human CLCA2 mediate lung metastasis. J Biol Chem. 2001 July 6;276(27):25438-46.

Borradori, L., and Sonnenberg, A. (1999). Structure and function of hemidesmosomes: more than simple adhesion complexes. J. Invest. Dermatol. 112, 411-418.

Chatterjee, M., Stuhmer, T., Herrmann, P., Bommert, K., Dorken, B., and Bargou, R. C. (2004). Combined disruption of both the MEK/ERK and the IL-6R/STAT3 pathways is required to induce apoptosis of multiple myeloma cells in the presence of bone marrow stromal cells. Blood 104, 712-3721.

Dans, M., Gagnoux-Palacios, L., Blaikie, P., Klein, S., Mariotti, A., and Giancotti, F. G. (2001). Tyrosine phosphorylation of the b4 integrin cytoplasmic domain mediates Shc signaling to extracellular signal-regulated kinase and antagonizes formation of hemidesmosomes. J. Biol. Chem. 276, 1494-1502.

de Bono, J. S., and E. K. Rowinsky. 2002. The ErbB receptor family: a therapeutic target for cancer. Trends Mol Med. 8:S19-26.

Fuchs, E., Dowling, J., Segre, J., Lo, S. H., and Yu, Q. C. (1997). Integrators of epidermal growth and differentiation: distinct functions for b1 and b4 integrins. Curr. Opin. Genet. Dev. 7, 672-682.

Gagnoux-Palacios, L., Dans, M., van't Hof, W., Mariotti, A., Meneguzzi, G., Resh M. D., and Giancotti, F. G. (2003). Integrin a6b4 signaling requires compartmentalization in lipid rafts. J. Cell Biol. 162, 1189-1196.

Geiger, B., Bershadsky, A., Pankov, R., and Yamada, K. M. (2001). Transmembrane crosstalk between the extracellular matrix and the cytoskeleton. Nat. Rev. Mol. Cell Biol. 2, 793-805.

Giancotti, F. G., and Tarone, G. (2003). Positional Control of Cell Fate through Joint Integrin/Receptor Protein Tyrosine Kinase Signaling. Ann. Rev. Cell Dev. Biol. 19:173-206.

Giancotti, F. G., and Ruoslahti, E. (1999). Integrin signaling. Science 285, 1028-1032.

Hynes, R. O. (2002). A reevaluation of integrins as regulators of angiogenesis. Nat. Med. 8, 918-921.

Hynes, R. O. (2003). Metastatic potential: generic predisposition of the primary tumor or rare, metastatic variants-or both? Cell 113 821-823.

Mainiero, F., Murgia, C., Wary, K. K., Curatola, A. M., Pepe, A., Blumemberg, M., Westwick, J. K., Der, C. J., and Giancotti, F. G. (1997). The coupling of a6b4 integrin to Ras-MAP kinase pathways mediated by Shc controls keratinocyte proliferation. EMBO J. 16, 2365-2375.

Mainiero, F., Pepe, A., Wary, K. K., Spinardi, L., Mohammadi, M., Schlessinger, J., and Giancotti, F. G. (1995). Signal transduction by the a6b4 integrin: distinct b4 subunit sites mediate recruitment of Shc/Grb2 and association with the cytoskeleton of hemidesmosomes. EMBO J. 14, 4470-4481.

Miranti, C. K., and Brugge, J. S. (2002). Sensing the environment: ahistorical perspective on integrin signal transduction. Nat. Cell Biol. 4, E83-90.

Murgia, C., Blaikie, P., Kim, N., Dans, M., Petrie, H. T., and Giancotti, F. G. (1998). Cell cycle and adhesion defects in mice carrying a targeted deletion of the integrin b4 cytoplasmic domain. EMBO J. 17, 3940-3951.

Natali P G, Nicotra M R, Botti C, Mottolese M, Bigotti A, Segatto Oanges in expression of alpha 6/beta 4 integrin heterodimer in primary and metastatic breast cancer. Br J Cancer. 1992 August;66(2):318-22

Pellegrini, G., De Luca, M., Orecchia, G., Balzac, F., Cremona, O. Ch., Savoia, P., Cancedda, R., and Marchisio, P. C. (1992). Expression, topography, and function of integrin receptors are severely altered in keratinocytes from involved and uninvolved psoriatic skin. J. Clin. Invest. 89, 1783-1795.

Schlaepfer, D. D., and Hunter, T. (1998). Integrin signaling and tyrosine phosphorylation: just the FAKs? Trends Cell Biol. 8, 151-157.

Shaw, L. M. (2001). Identification of Insulin Receptor Substrate 1 (IRS-1) and IRS-2 as Signaling Intermediates in the a6b4 Integrin-Dependent Activation of PI3-K and Promotion of Invasion. Mol. Cell Biol. 21, 5082-5093.

Shaw, L. M., Rabinovitz, I., Wang, H. H., Toker, A., and Mercurio, A. M. (1997). Activation of PI3-K by the a6b4 integrin promotes carcinoma invasion. Cell 91, 949-960.

Slamon D J, Clark G M, Wong S G, Levin W J, Ullrich A, McGuire W L. Human breast cancer: correlation of relapse and survival with amplification of the HER-2/neu oncogene. Science. 1987 January 9;235(4785):177-82.

Spinardi, L., Einheber, S., Cullen, T., Milner, T. A., and Giancotti, F. G. (1995). A recombinant tail-less integrin b4 subunit disrupts hemidesmosomes, but does not suppress a6b4-mediated cell adhesion to laminins. J. Cell Biol. 129, 473-487.

Tagliabue E, Ghirelli C, Squicciarini P, Aiello P, Colnaghi M I, Menard S.

Prognostic value of alpha 6 beta 4 integrin expression in breast carcinomas is affected by laminin production from tumor cells. Clin Cancer Res. 1998 Feb;4(2):407-10.

Trusolino, L., Bertotti, A., and Comoglio, P. M. (2001). A signaling adapter function for a6b4 integrin in the control of HGF-dependent invasive growth. Cell 107, 643-654.

Wary, K. K., Mariotti, A., Zurzolo, C., and Giancotti, F. G. (1998). A requirement for caveolin-1 and associated kinase Fyn in integrin signaling and anchorage-dependent cell growth. Cell 94, 625-634.

Weaver, V. M., Lelievre, S., Lakins, J. N., Chrenek, M. A., Jones, J. C., Giancotti, F., Werb, Z., and Bissell, M. J. (2002). b4 integrin-dependent formation of polarized three-dimensional architecture confers resistance to apoptosis in normal and malignant mammary epithelium. Cancer Cell 2, 205-216.

1. A method for inhibition of initiation of primary or metastatic tumor growth in an individual suffering from or at risk for a tumor type that expresses a6b4 integrin, comprising the steps of administering to the individual a therapeutic agent effective to reduce the amount of active a6b4 integrin at least in a portion of the individual where initiation of primary or metastatic tumor growth may occur by targeting the beta 4 portion of the integrin.

2. The method of claim 1, wherein the individual is human.

3. The method of claim 2, wherein the therapeutic agent is an antibody.

4. The method of claim 2, wherein the therapeutic agent is an antisense oligonucleotide.

5. The method of claim 2, wherein the therapeutic agent is an RNAi species.

6. The method of claim 2, wherein the individual is suffering from or at risk for a tumor type selected from the group consisting breast; and prostate cancers.

7. The method of claim 6, further comprising the step of administering to the individual an inhibitor of a receptor protein tyrosine kinase.

8-12. (canceled)

13. The method of claim 7, wherein the receptor protein tyrosine kinase is selected from the group consisting of ErbB2, EGF-R, Met and Ron.

14. The method of claim 2, further comprising the step of administering to the individual an inhibitor of a receptor protein tyrosine kinase.

15. The method of claim 14, wherein the receptor protein tyrosine kinase is selected from the group consisting of ErbB2, EGF-R, Met and Ron.

16. A method for inhibition of initiation of primary or metastatic tumor growth in an individual suffering from or at risk for a tumor type that expresses a6b4 integrin, comprising administering to the individual a therapeutic agent effective to reduce the amount of active a6b4 integrin at least in a portion of the individual where initiation of primary or metastatic tumor growth may occur by targeting the beta 4 portion of the integrin, wherein the tumor expresses an amplified or activated version of a receptor protein kinase.

17. The method of claim 16, wherein the receptor protein kinase is selected from the group consisting of erbB2, EGF-R, Met and Ron.

18. The method of claim 16, wherein the individual is human.

19. The method of claim 18, wherein the tumor is breast cancer or prostate cancer.

20. The method of claim 18, wherein the therapeutic agent is an antibody.

21. The method of claim 18, wherein the therapeutic agent is an antisense oligonucleotide.

22. The method of claim 18, wherein the therapeutic agent is an RNAi species.

23. The method of claim 18, further comprising the step of administering to the individual an inhibitor of a receptor protein tyrosine kinase.

24. The method of claim 23, wherein the receptor protein tyrosine kinase is selected from the group consisting of erbB2, EGF-R, Met and Ron.
